(19) United States
(12) Patent Application Publication
(10) Pub. No.: US 2025/0021693 A1
(43) Pub. Date: Jan. 16, 2025

(54) TECHNIQUES FOR DE-IDENTIFYING SENSOR DATA CAPTURED IN A MEDICAL FACILITY ROOM

(71) Applicant: EXIN HEALTH, INC., Palo Alto, CA (US)

(72) Inventors: Hasan ERTAS, Palo Alto, CA (US); Metin SEZGIN, Palo Alto, CA (US); Ugur ACAR, Palo Alto, CA (US)

(21) Appl. No.: 18/767,802

(22) Filed: Jul. 9, 2024

Related U.S. Application Data
(60) Provisional application No. 63/525,886, filed on Jul. 10, 2023.

(51) Int. Cl. G06F 21/62; G16H 10/60

(52) U.S. Cl. CPC G06F 21/6254; G16H 10/60

(57) ABSTRACT

Various embodiments set forth systems and techniques for de-identifying data captured in a medical facility room. In some embodiments, an edge computing device (ECD) operating within a security boundary of a medical facility receives a set of streams of sensor data captured in set of medical facility rooms in the medical facility. For each stream of the set of streams of sensor data, the ECD generates a de-identified data stream at least based on redacting from the stream information that identifies one or more persons, and generates identity-neutral metadata associated with the de-identified data stream at least based on extracting from the stream information that describes the identified persons in a manner that does not reveal the identity of the identified persons. The ECD transmits the de-identified data streams and the associated identity-neutral metadata for further processing outside of the security boundary of the medical facility.

### CROSS-REFERENCE TO RELATED APPLICATIONS

This application claims priority benefit of United States Provisional patent application titled “TECHNIQUES USING ARTIFICIAL INTELLIGENCE AND COMPUTER VISION TO OPTIMIZE OPERATIONAL EFFICIENCY AND CLINICAL OUTCOMES IN A SURGICAL ENVIRONMENT,” Ser. No. 63/525,886, filed Jul. 10, 2023. The subject matter of this related application is hereby incorporated herein by reference.

### BACKGROUND

### Technical Field

Embodiments of the present disclosure relate generally to computer science and data security and, more specifically, to techniques for safeguarding digital data collected in a healthcare setting.

### Description of the Related Art

Digital data collected in a healthcare setting (e.g., a medical facility room) includes sensitive and confidential information. Use of such data is often subject to privacy considerations, laws and regulations, such as Health Insurance Portability and Accountability Act (HIPAA). Some current techniques for safeguarding the privacy of such data resort to keeping the use of such data local to where the data is originally collected (e.g., a medical facility). However, the lack of data diversity in systems that operate only on locally stored data prevents such systems from producing rich data analytics and/or training robust predictive models (e.g., artificial intelligence (AI)/machine learning (ML) models). Furthermore, such systems often cannot take advantage of large scale compute resources, such as cloud computing services, which leads to these systems being non-performant, inefficient, or difficult to maintain.

Some other current techniques for safeguarding the privacy of such data resort to redacting the sensitive and confidential information from the raw data before transmitting the data for further processing in a less controlled environment. However, such redaction may remove important data features or relationship(s) between the redacted information and the remaining data. The removal of important data features through the redaction process often interferes with the effective operation of downstream applications that derive meaningful data analytics and/or training robust predictive models based on the raw data.

As the foregoing illustrates, what is needed in the art are more effective techniques for safeguarding the privacy of healthcare data while allow for using such data to produce rich data analytics and/or training robust predictive models.

### SUMMARY

One embodiment of the present disclosure sets forth a computer-implemented method for de-identifying data captured in medical facility rooms. The method includes receiving a set of streams of sensor data captured in set of medical facility rooms in a medical facility. The method further includes for each stream of the set of streams of sensor data, generating a de-identified data stream at least based on redacting from the stream information that identifies one or more persons and generating identity-neutral metadata associated with the de-identified data stream at least based on extracting from the stream information that describes the identified persons in a manner that does not reveal the identity of the identified persons. In addition, the method includes transmitting the de-identified data streams and the associated identity-neutral metadata for further processing.

Other embodiments of the present disclosure include, without limitation, one or more computer-readable media including instructions for performing one or more aspects of the disclosed techniques as well as one or more computing systems for performing one or more aspects of the disclosed techniques.

At least one technical advantage of the disclosed techniques relative to the prior art is that the disclosed techniques can be effectively utilized to safeguard the privacy of healthcare data without sacrificing the ability to generate rich data analytics and/or train robust predictive models based on that data or vice versa. In particular, given a data stream captured in a medical facility room, the disclosed techniques can redact confidential and sensitive information from the data stream while at the same time generating identity-neutral metadata associated with the data stream. Such identity-neutral metadata preserves important data features in the data stream without revealing the underlying confidential and sensitive information in the data stream. Such a technique enables further processing at downstream applications to train predictive models (e.g., Al/ML models) and extract rich analytics. These technical advantages represent one or more technological improvements over prior art approaches.

### BRIEF DESCRIPTION OF THE DRAWINGS

So that the manner in which the above recited features of the various embodiments can be understood in detail, a more particular description of the inventive concepts, briefly summarized above, may be had by reference to various embodiments, some of which are illustrated in the appended drawings. It is to be noted, however, that the appended drawings illustrate only typical embodiments of the inventive concepts and are therefore not to be considered limiting of scope in any way, and that there are other equally effective embodiments.

FIG. 1 illustrates a system configured to implement one or more aspects of the present disclosure;

FIG. 2 is a more detailed illustration of an exemplar medical facility room (MFR) 102 of FIG. 1, according to various embodiments;

FIG. 3 is a more detailed illustration of an exemplar edge computing device (ECD) 120 of FIG. 1, according to various embodiments;

FIG. 4 is a more detailed illustration of an exemplar cloud computing device (CCD) 140 of FIG. 1, according to various embodiments; and

FIG. 5 is a flow diagram of method steps for ECD 120 in FIGS. 1-3, according to various embodiments.

### DETAILED DESCRIPTION

In the following description, numerous specific details are set forth to provide a more thorough understanding of the various embodiments. However, it will be apparent to one of skilled in the art that the inventive concepts may be practiced without one or more of these specific details.

### System Overview

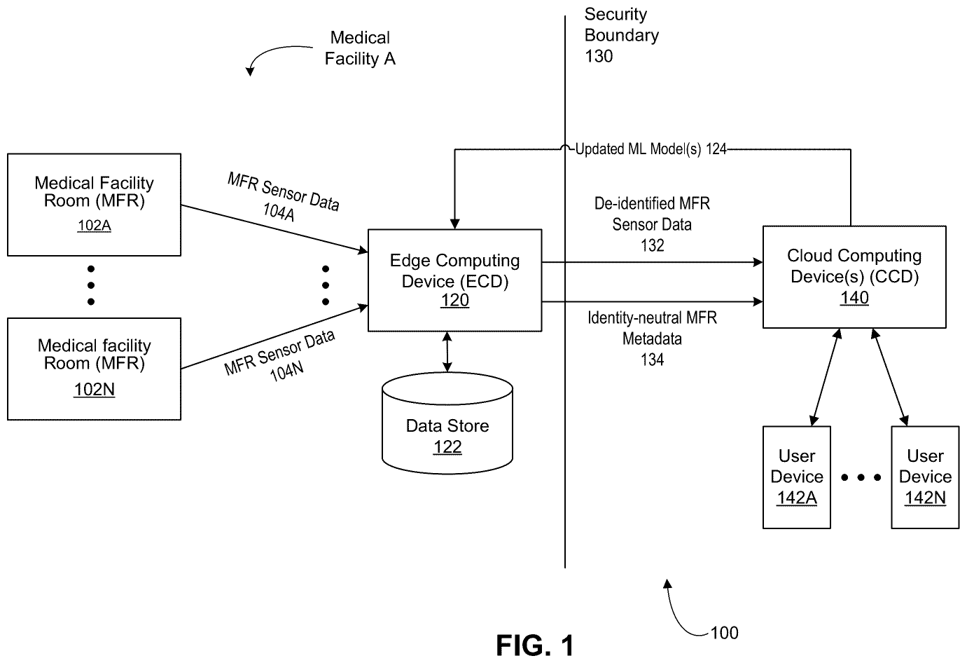

FIG. 1 illustrates a system 100 that implements one or more aspects of the various embodiments. As shown, the system 100 includes an edge computing device (ECD) 120, a data store 122, and cloud computing device(s) (CCD) 140 in communication with the ECD 120 over a network (not shown), which can be a wide area network (WAN) such as the Internet or any other suitable network. The ECD 120 is also in communication with one or more medical facility rooms (MFR) 102A-N (which are collectively referred to herein as “MFRs 102” and individually referred to herein as “an MFR 102”), such as an operating room, an intensive care unit (ICU), an emergency care room, in a medical facility A, such as a hospital. The MFRs 102A-N and the ECD 120 are within a security boundary 130 (e.g., a firewall) of the medical facility A, in contrast to the CCD 140 that is outside the security boundary 130.

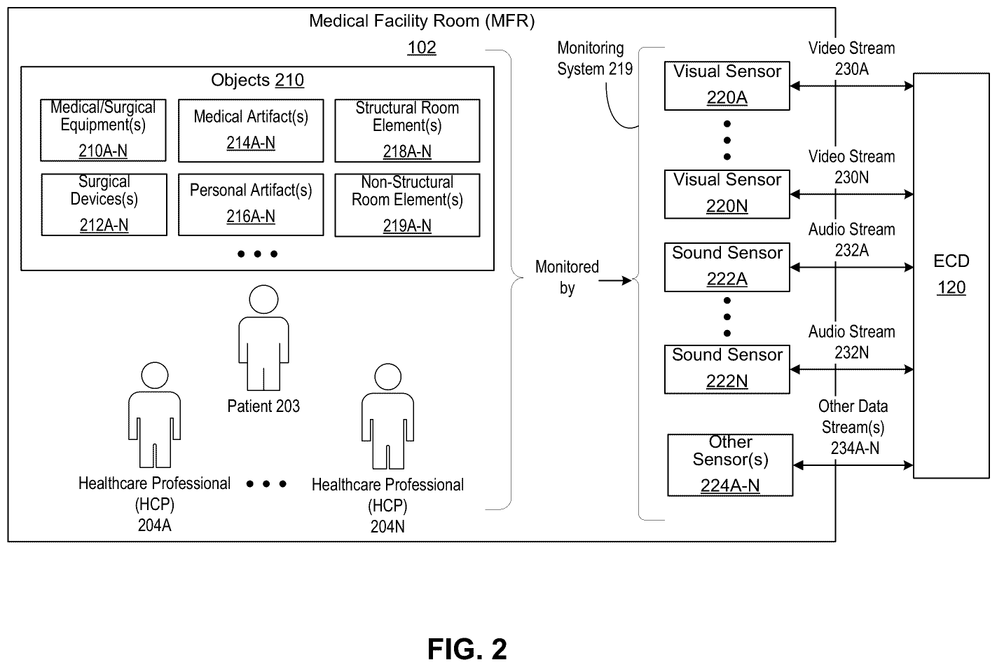

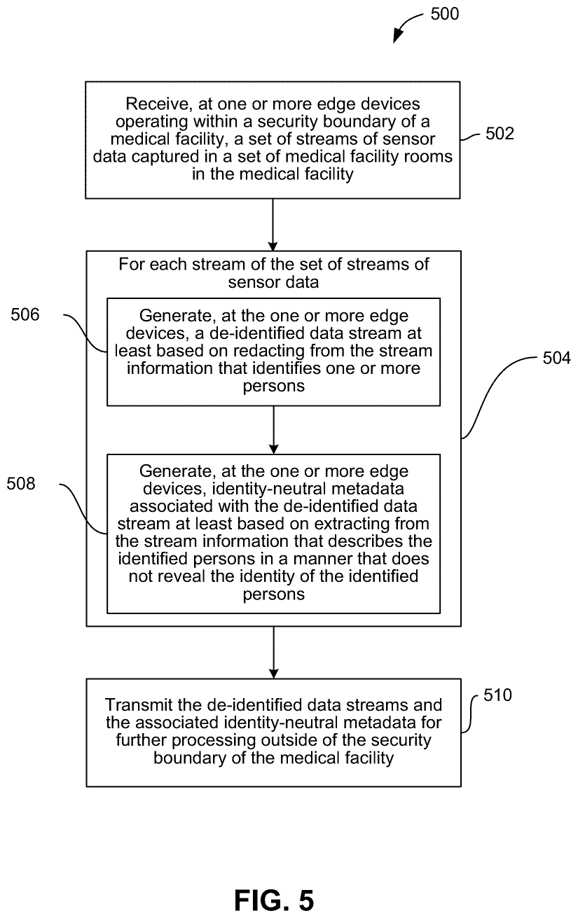

In operation, the ECD 120 receives as input MFR sensor data 104A-N from the MFRs 102A-N, respectively. Examples of MFR sensor data 104A-N include, but is not limited to, visual sensor data, audio sensor data, temperature sensor data. The MFR sensor data 104A-N are captured by various types of sensors in the MFRs 102A-N, respectively. The MFR sensor data 104A-N are discussed in greater detail below in conjunction with FIGS. 2 and 5.

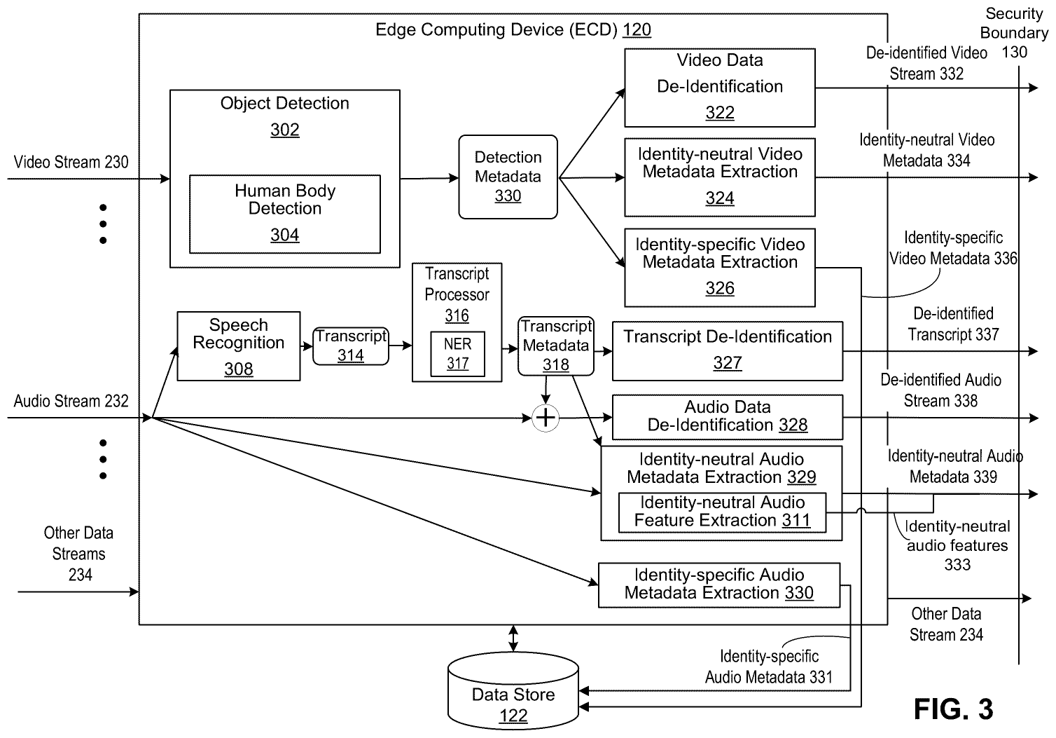

Given the MFR sensor data 104A-N, the ECD 120 performs local processing of the MFR sensor data 104A-N within the security boundary 130 to generate de-identified MFR sensor data 132 and identity-neutral MFR metadata 134 as output. In various embodiments, to de-identify MFR sensor data 104A-N, the ECD 120 redacts information from the MFR sensor data 104A-N that can be used to identify a person. Such information includes, but is not limited to, personal identifying information (PII) (e.g., an SSN, a person name, and the face of a person. In some embodiments, the ECD 120 redacts information from the MFR sensor data 104A-N that cannot be used to identify a person but is sensitive in nature. Such information includes, but is not limited to, an image of genitalia. In various embodiments, to generate the identity-neutral MFR metadata 134, the ECD 120 extracts from the MFR sensor data 104A-N information that describes person(s) and/or event(s) related to the person(s) in a way that the underlying identity of a person cannot be discerned. In some embodiments, the redaction and/or extraction operations on the MFR sensor data 104A-N can be implemented using one or more ML models. The de-identified MFR sensor data 132 and the identity-neutral MFR metadata 134, as well as the redaction and extraction operations are discussed in greater detail below in conjunction with FIGS. 3 and 5. Once the local processing completes, the ECD 120 transmits the de-identified MFR sensor data 132 and the identity-neutral MFR metadata 134 to the CCD 140 for further processing.

In some embodiments, the local processing can also include generating identity-specific metadata (not shown). The ECD 120 extracts information from the MFR sensor data 104-A-N that describes person(s) and/or event(s) related the person(s) in a way that either directly or with further processing allows the underlying identity of a person to be discerned. Thus, for the purpose of safeguarding the confidentiality of such data, the identify-specific metadata is stored locally in the data store 122 and remains within the security boundary 130. The identity-specific metadata is discussed in greater detail below in conjunction with FIGS. 3 and 5.

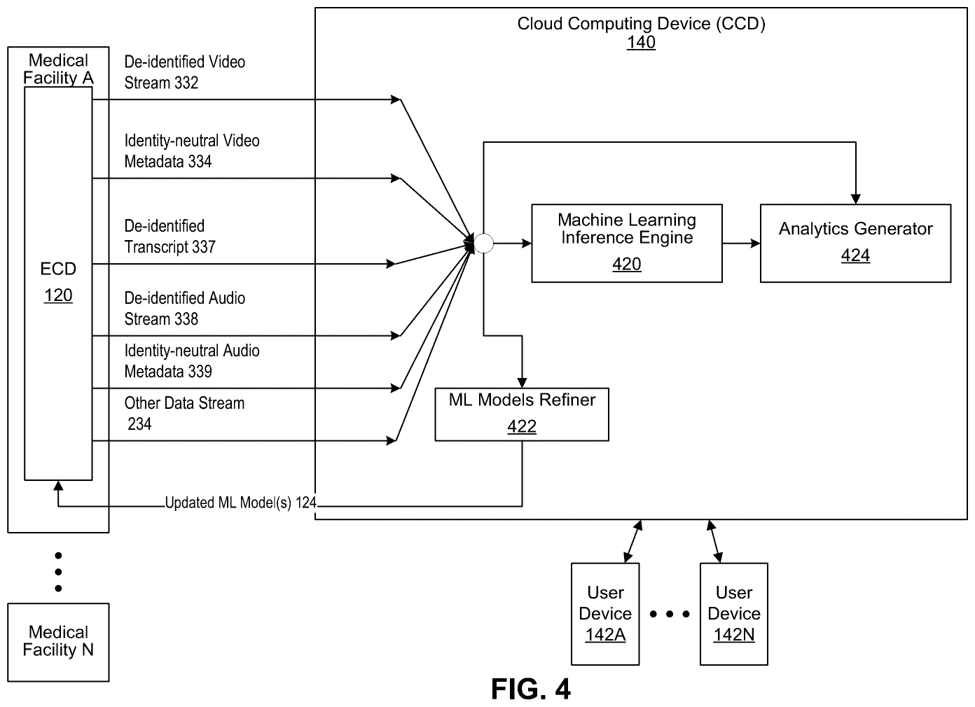

Given the de-identified MFR sensor data 132 and the identity-neutral MFR metadata 134 transmitted from the ECD 120, the CCD 140 performs further processing of such data. In some embodiments, the CCD 140 generates data analytics based on such data. In some embodiments, the CCD 140 generates inference(s) from such data and generates data analytics based on the inference(s). In some embodiments, the CCD 140 generates data analytics from a combination of such data and the inferences. Such data analytics are made available to one or more user devices (illustrated as user devices 142A-N) that are in communication with the CCD 140. In some embodiments, the CCD 140 trains or updates trained ML model(s) (shown as updated ML model(s) 124) based on the data received from the ECD 120. Such models can be used in the redaction and/or extraction operations of the ECD 120, as described above. The operations of the CCD 140 are discussed in greater detail below in conjunction with FIGS. 4 and 5.

It will be appreciated that the system 100 shown herein is illustrative and that variations and modifications are possible. For example, the number of ECDs 120, data stores 120, and CCDs 120 can be modified as desired. As an example, in various embodiments, medical facility rooms and edge computing devices in other medical facilities operating within their respective security boundaries communicate with CCD 140 in a manner similar to the MFRs 102 and the ECD 120. Further, the medical facility A is also illustrative and can be any healthcare provider facility that provides one or more medical facility rooms.

### Medical Facility Room

FIG. 2 is a more detailed illustration of one exemplar medical facility room 102 of FIG. 1, according to various embodiments. As shown in FIG. 2, an MFR 102 includes one or more objects 210 (which are individually referred to herein as “an object 210”). As shown, objects 210 can include one or more surgical equipments 210A-N (e.g., surgical bed, surgical monitor(s), etc.), one or more surgical devices 212A-N (e.g., surgical scissors, surgical staplers, graspers, trocars, forceps, etc.), one or more medical artifacts 214A-N (e.g., surgical trays, skin prep supplies, surgical drapes, etc.), one or more personal artifacts 216A-N (e.g., clothing, objects of personal adornment, eyeglasses, identification (ID) badges, etc.), one or more structural room elements 218A-N (e.g., floor, walls, ceiling, column(s), etc.), one or more non-structural room elements 219A-N (e.g., window(s), door(s), light fixture, etc.), one or more clean supply items (e.g., mops, trash bags, etc.), and the like.

The MFR 102 can also include one or more healthcare professionals (HCP) 204A-N (which are collectively referred to herein as “HCPs 204” and individually referred to herein as “an HCP 204”). The HCPs 204 can include healthcare professionals in various roles, such as nurse(s) (e.g., scrub nurse, circulating nurse, bedside assistant(s), surgeon(s)), anesthesiologist(s), and the like. The MFR 102 can also include one or more patients 203 (which are collectively referred to herein as “patients 203” and individually referred to herein as “a patient 203”) (e.g., when a surgical procedure is in session). In some cases, the MFR 102 can include other personnel (not shown), such as facility management staff, cleaning crew, etc.

The MFR 102 includes a monitoring system 219 that can include one or more visual sensors 220A-N (which are collectively referred to herein as “visual sensors 220” and individually referred to herein as “a visual sensor 220”). Visual sensors 220 can be video cameras (e.g., surveillance cameras), surgical cameras, three-dimensional (3D) cameras, and the like. The monitoring system 219 can also include one or more sound sensors 222A-N (which are collectively referred to herein as “sound sensors 222” and individually referred to herein as “a sound sensor 222”). Sound sensors 222 can include microphones, audio recorders, speakers, and the like. The monitoring system 219 can also include one or more other sensors 224A-N. The other sensors 224A-N can include temperature sensor(s), humidity sensor(s), vibration sensor(s), ambient light sensor(s), radio frequency identification (RFID) device(s), and devices with Bluetooth capabilities.

In operation, the objects 210, HCPs 204, patient 203, other personnel, and/or the interactions between them are monitored by the monitoring system 219. As a result, various data streams that capture the physical presence and movements of these various entities are generated. In some embodiments, visual sensors 220A-N respectively generate video streams 230A-N (which are collectively referred to herein as “video streams 230” or “video data streams 230,” and individually referred to herein as “a video stream 232” or “a video data stream 230”). The video streams 230 can capture the physical presence of an HCP 204 or patient 203 or other personnel and can include the imagery of the body, the body's location in the MFR 102, the body's location relative to other HCP(s) 204 or patient 203 or other personnel, the personal artifacts on the body, the medical artifacts on the body, etc. The video streams 230 can also capture the physical presence of an object 210, such as its imagery, its absolute location in the MFR 102, its location relative to other object(s) and/or HCP(s) 204 or patient 203 or other personnel, etc.

The video streams 230 can also capture the movements of an HCP 204 or the patient 203 or other personnel as the person moves to a different location in the MFR 102 or moves a part of the body (e.g., a hand, the head, etc.) while staying at the same location in the MFR 102. The video streams 230 can also capture the movements of an object 210 caused by an HCP 204 or the patient 203 or other personnel. In a first example, the movement of a surgical device 212 being held by a surgeon can be captured. In a second example, the movement of a surgical drape being put on a patient by an HCP 204 can be captured. In a third example, the movement of the door of the MFR 102 can be captured as an HCP 204 pushes the door to enter the MFR 102. In a fourth example, the movement of a surgical monitor can be captured as an HCP 204 positions the monitor as part of setting up the MFR 102 for a surgical procedure and/or adjusts the position of the surgical monitor during a surgical procedure. It should be understood that these examples of movement are illustrative and that the types of the movements of the objects 102 and HCPs 204, patient 203 or other personnel in the MFR 102 are innumerable.

In some embodiments, sound sensors 222A-N respectively generate audio streams 232A-N (which are collectively referred to herein as “audio streams 232” or “audio data streams 232,” and individually referred to herein as “an audio stream 232” or “an audio data stream 232”). The audio streams 232 can capture the voice and/or movements of an HCP 204 or patient 203 or other personnel. The audio streams 232 can also capture the sounds from the movement of the medical/surgical equipments 210, alarms from the medical/surgical equipments 210 (e.g., from a surgical monitor), the clanging and dropping of surgical devices 212, etc. It should be understood that these examples of sounds are illustrative and that the type of sounds in the MFR 102 are innumerable. In some embodiments, other sensors 224 generate various data streams that capture other information in the MFR 102 such as the temperature, ambient light, vibrations.

As shown, the video streams 230, audio streams 232, and/or other data streams 234 are transmitted to the ECD 120. To safeguard the confidentiality of these data streams, these data streams can be encrypted before transmission. In some embodiments, these data streams are transmitted in real-time or near real-time (e.g., transmitted as soon as they are captured by the respective sensors or upon request by an application that requires such data streams). In some other embodiments, these data streams are transmitted periodically (e.g., stored at first in the on-device storage in the respective sensors and transmitted after the on-device storage is full or nearly full). It should be appreciated that any other suitable transmission configuration(s) can be implemented.

In some embodiments, the ECD 120 and/or CCD 140 can transmit one or more data streams back to the MFR 102, e.g., based on data processing performed on the ECD 120 and the CCD 140. For instance, the ECD 120 may transmit voice message(s) and/or voice warning(s) generated by the ECD 120 and/or CCD 140 to one or more sound sensors 222 or other devices to be played back. As another example, the ECD 120 may transmit visual messages and/or warnings generated by the ECD 120 and/or CCD 140 to visual sensors 220 or other devices (e.g., actuators, tablets, displays) to be displayed. An yet another example, the ECD 120 may transmit message(s) and/or control signal(s) to other sensors 224.

### Edge Computing Device

FIG. 3 is a more detailed illustration of an exemplar edge computing device 120 of FIG. 1, according to various embodiments. As shown, the ECD 120 includes various software applications (or any other forms of computer-executable instructions) (e.g., stored in a system memory (not shown)). Such software applications can include an object detection 320, video data de-identification 322, identity-neutral video metadata extraction 324, identity-specific video metadata extraction 326, speech recognition 308, transcript processor 316 (which can include named entity recognition (NER) 316), transcript de-identification 327, audio data de-identification 328, identity-neutral audio metadata extraction 329, and identity-specific audio metadata extraction 330.

In operation, any of such software applications above can execute on one or more processors (not shown) (e.g., CPU(s) or GPU(s)) to perform one or more operations. For example, as shown, the object detection 302 receives as input a video stream 230 transmitted from the MFR 102, as described in FIG. 2, and can detect object(s) (e.g., the objects 210 shown in FIG. 2) and human body(s) (e.g., an HCP 204 or a patient 203 or other personnel) in the video stream 230. The object detection 302 can be implemented using one or more object detection predictive algorithms, such as predictive ML models like YOLO (You Only Look Once), R-CNN (Region-based Convolutional Neural Networks), and the like. As shown, in some embodiments, the object detection 302 can include a human body detection 304 that is configured to be part of the algorithm(s) of the object detection 302 to detect human bodies, such as bodies of healthcare professionals and/or patients. Such configuration can be based on training the above-mentioned object detection algorithm(s) with images of HCPs in various types of uniforms and patients in various types of medical clothing (e.g., patient gowns, surgical drapes). Such configuration can also be based on training the above-mentioned object detection algorithm(s) with images that only include a part of a human body (e.g., a hand, an arm, a leg, etc.), including sensitive body part(s) (e.g., genitalia).

In some embodiments, the object detection 302 and the human body detection 304 can be implemented separately from each other. In such a case, the object detection 302 and the human body detection 304 each independently receives as input the video stream 230 and performs object detection and human body detection, respectively.

When the object detection 302 detects an object in a frame of the video stream 230, the object detection 302 generates as output detection metadata 330 (which is herein used to refer to, for example, metadata for a single detected object or human body, or collectively the metadata for a subset or all of the detected objects and/or human bodies) that describes the detected object. Such detection metadata 330 can indicate the boundary of the detected object in the frame (e.g., in a format of pixel coordinates).

Such detection metadata 330 can also indicate what is detected in the boundary. For example, the detection metadata 330 can indicate whether the detected object is a human or, more specifically, a specific persona, such as a patient or an HCP role, as described above. Furthermore, the detection metadata 330 can indicate whether the detected object is a specific body part (e.g., a face, genitalia, a hand, an arm, a leg, a torso, etc.). As another example, the detection metadata 330 can indicate the type of a detected object (e.g., any of the objects 210 shown in FIG. 2). Further, when the object detection 302 detects text in the image of a detected object (e.g., a whiteboard, a monitor (e.g., an LCD monitor), a medical chart, a name tag, a badge, etc.), the detection metadata 330 can indicate that the detected object includes text and what the detected text is.

As shown, the video data de-identification 322 receives the detection metadata 330 as input. Given the detection metadata 330, the video data de-identification 322 can redact information from the video stream 230 that can be used to identify a person (directly or with some processing) and/or sensitive information (regardless of if it cannot be used to identify a person). In some embodiments, the video data de-identification 322 redacts the entirety of a human body. In some embodiments, the video data de-identification 322 redacts an enlarged area surrounding the human body. In some embodiments, the video data de-identification 322 redacts the part(s) of a human body that can easily be used to identify a person (e.g., a face) and leaves the rest of the body intact. In some embodiments, the video data de-identification 322 redacts the sensitive part(s) of a human body (e.g., genitalia) and the part(s) of a human body that can easily be used to identify a person, and leaves the rest of the body intact.

Given the detection metadata 330, the video data de-identification 322 can also redact from the video stream 230 detected text that includes PII information, such as a person name, social security number (SSN), date of birth (DOB), employee identifier, etc. To redact information from a frame, redaction operation at the video data de-identification 322 can be implemented in various ways, such as deleting pixels, applying a blurring or mosaic filter/mask, replacing the color codes of the original pixels with a dark color code (e.g., black), editing pixels (e.g., in painting using generative ML model(s)), and the like. As a result of such redactions, the video stream 230 is transformed to the de-identified video stream 332. As shown, the de-identified video stream 332 is transmitted outside the security boundary 130 for further processing (e.g., to the cloud computing device(s) 140 (shown later in FIG. 4)). In some embodiments, the detection metadata 330 is also transmitted along with the de-identified video stream 332 outside the security boundary 130 for further processing at the cloud computing device(s) 140.

In some embodiments, the video data de-identification 322 can perform de-identification of objects and human bodies using cross-stream information. For example, the video data de-identification 322 can use object(s) identified by a first video stream 230 for redaction to identify the same object(s) in a second video stream 230 and accordingly redact such object(s) from the second video stream 230.

As shown, the identity-neutral video metadata extraction 324 also receives the detection metadata 330 as input and outputs identity-neutral video metadata 334. As discussed above, the identity-neutral video metadata 334 can include metadata describing any persons that were identified in the video stream in an identity-neutral way, such that the underlying identity of the person cannot be discerned from the identity-neutral metadata. In some embodiments, given the detection metadata 330 of the detected body of an HCP or patient or other personnel in a video frame of the video stream 230, the identity-neutral video metadata extraction 324 can generate metadata that includes the pixel location information for the major joints (e.g., shoulder(s), elbow(s), wrist(s), hip(s), knee(s), ankle(s)) of the detected body and an associated color code at these pixel location(s), and pixel location information of a straight line that connect these major joints. Accordingly, by combining such metadata with the corresponding de-identified video frame, a downstream application can render a “skeleton” representation of the detected body. In such a manner, such metadata enables a representation of a person that indicates a person without revealing the identity of the person. Additionally or alternatively, the identity-neutral video metadata extraction 324 can generate metadata that indicates the persona of the detected person (a patient or an HCP role or other personnel's role, as described above). In some cases, when only the most identity-revealing and sensitive body part(s) (e.g., the face and genitalia) are redacted, such metadata is not generated. In such cases, because the unredacted portion(s) of the detect body provides sufficient information that indicates a person, such metadata is not needed.

In some embodiments, given the detection metadata 330 of the detected body of an HCP or patient or other personnel in a video frame of the video stream 230, the identity-neutral video metadata extraction 324 can generate metadata describing event(s) that occurred in the video frame between different identified persons and/or between identified person(s) and identified objects. To generate such metadata, the identity-neutral video metadata extraction 324 can be implemented using AI/ML model(s) (e.g., discriminative and/or generative ML model(s)). In a first example, given the detection metadata 330 indicating a detected patient and detected medical artifact(s) (e.g., a surgical drape) overlapping with the location of the patient's body, the identity-neutral video metadata extraction 324 generates an event of a patient wearing a surgical drape. In a second example, given the detection metadata 330 indicating a detected patient and detected medical artifact(s) (e.g., an inhalation mask) overlapping with the location of the patient's face, the identity-neutral video metadata extraction 324 generates an event of a patient wearing an inhalation mask. In a third example, given the detection metadata 330 indicating a detected HCP and the face of the detected HCP not being covered with a face mask or the head of the detected HCP not being covered with a head cover, the identity-neutral video metadata extraction 324 generates an event that indicates such is the case. In a fourth example, given the detection metadata 330 indicating a surgeon holding a surgical instrument, the identity-neutral video metadata extraction 324 generates an event that indicates such is the case.

In some embodiments, given the detection metadata 330 of the detected body of an HCP or patient or other personnel in a series of video frames of the video stream 230, the identity-neutral video metadata extraction 324 can generate metadata that represents various types of events and/or actions captured in the series of video frames. In a first example, given the detection metadata 330 indicating a rubbing motion by a HCP over a patient and a portion of the patient's body being in a color different from the color of the bare skin, the identity-neutral video metadata extraction 324 generates an event/action that indicates a patient's skin is being prepared for a surgical procedure. In a second example, given the detection metadata 330 indicating that two HCPs are facing each other and the lips of both of the HCPs are moving, the identity-neutral video metadata extraction 324 generates an event indicating these HCPs are having a conversation. In a third example, given the detection metadata 330 indicating the door of the MFR 102 changing from a closed position to an open position and to a closed position again, the identity-neutral video metadata extraction 324 generates an event indicating that someone has entered or left the MFR 102. It should be understood that the examples of events/actions above are illustrative and that the events in the MFR 102 are innumerable.

In some embodiments, the identity-neutral video metadata extraction 324 can associate the extracted identity-neutral video metadata 334 with one or more of the following information: data identifying the type of a given surgical procedure, data identifying the MFR 102, data identifying the medical facility A.

As shown, the identity-neutral video metadata extraction 324 transmits identity-neutral neutral video metadata 334 outside the security boundary 130 for further processing (e.g., to the cloud computing device(s) 140).

As shown, the identity-specific video metadata extraction 326 also receives the detection metadata 330 as input. Given the detection metadata 330 of the HCP 204s in a series of video frames in the video stream 230, the identity-specific video metadata extraction 326 can generate various types of metadata about the HCP 204, such as a model that uniquely represents the HCP 204 and, in some embodiments, behavioral pattern(s) of the HCP 204.

In a first example, based on the images of detected HCPs 204 in a series of video frames in the video stream 230, the identity-specific video metadata extraction 326 can generate a model that uniquely represents a HCP 204 in the video stream 230 In contrast to the identity-neutral neutral video metadata 334, such model(s) of HCP 204 are not transmitted outside the security boundary 130 and are instead stored locally in the data store 122 within the security boundary 130, to safeguard the confidentiality of such data. Such model(s) of HCPs 204 can be used to re-identify the same HCPs 204 whose images are captured in the MFR 102 in the future.

Further, once such model(s) of HCPs 204 are available, given a video stream 230 captured in the future, the identity-specific video metadata extraction 326 can use the model(s) to generate metadata that describe the behavioral pattern(s) of a same HCP 204. In a first example, the detection metadata 330 generated from such a future video stream 230 can indicate a same surgeon interacting with a particular set of one or more surgical instruments during a particular type of surgical procedures, the order in which the surgeon interacts with the set of surgical instruments, and the duration of each interaction with a surgical instrument. The identity-specific video metadata extraction 326 can infer such interaction(s) with surgical instrument(s) from the movement of the surgeon's hand coinciding with the movement of a surgical instrument. Accordingly, the identity-specific video metadata extraction 326 can generate metadata that records such behavioral pattern(s) of the surgeon. In a second example, the detection metadata 330 can indicate a same surgeon interacting with a particular surgical equipment (e.g., a surgical monitor) during a particular type of surgical procedure, the frequency of the interaction(s), the duration of each interaction, etc. The identity-specific video metadata extraction 326 can infer such interaction(s) with the surgical equipment from the surgeon's hand overlapping with the surgical equipment or eye gaze in the direction of the surgical equipment. Accordingly, the identity-specific video metadata extraction 326 can generate metadata that records such behavioral pattern(s) of the surgeon. In a third example, the detection metadata 330 can indicate the movement(s) of one or more same HCPs 204 and/or their interaction(s) with detected object(s) before a patient enters the MFR 102. The identity-specific video metadata extraction 326 can infer from such movement(s) and/or interaction(s) that the HCP(s) 204 are performing various tasks preparing for a surgical procedure. Accordingly, the identity-specific video metadata extraction 326 can generate metadata that records information related to task(s) performed by the HCP(s) 204, such as the type of the task(s) (e.g., cleaning the floor, positioning the surgical equipments, setting up the surgical bed, etc.), including duration of the task(s), the order in which a series of tasks are performed, interaction(s) between the HCP(s) (e.g., conversation(s)) while they perform the tasks. It should be understood that these examples of identity-specific video metadata 336 above are illustrative and that the identity-specific video metadata 336 can record innumerable types of behavioral patterns of the HCPs 204 in the MFR 102.

In contrast to the identity-neutral neutral video metadata 334, the identity-specific video metadata 336 is not transmitted outside the security boundary 130 and is instead stored in the data store 122 within the security boundary 130, to safeguard the confidentiality of such data. Such locally stored identity-specific video metadata 336 can be further processed for a variety of local uses, such as generating report(s), data analytics, and/or operational recommendations to be used within the medical facility A.

As shown, the speech recognition 308 receives as input the audio stream 232 and outputs a transcript 314. The speech recognition 308 can be implemented using one or more automatic speech recognition (ASR) AI/ML models, such as a connectionist temporal classification (CTC) model, a sequence-to-sequence (Seq2Seq) model, and the like.

The transcript processor 316 receives as input the transcript 314 and outputs a transcript metadata 318. As part of the transcript processor 316, the NER 317 identifies and classifies entities in the transcript metadata 318 into categories, such as person names, organizations, locations, medical codes, SSNs, time or date expressions, etc. The NER 317 can be implemented using any suitable natural language processing (NLP) ML/AI model that identifies and classifies entities in text. Because the transcript 314 also includes timestamp information for the text, the transcript processor 316 can associate each identified entity with corresponding timestamps (e.g., start and end timestamps) while generating the transcript metadata 318.

The transcript de-identification 327 receives as input the transcript metadata 318 and outputs a de-identified transcript 337. More specifically, for each identified entity that include PII or sensitive information (e.g., a person name, SSN, DOB, time, date), the transcript de-identification 327 can redact that information (e.g., replacing the identified entity with special character(s) (e.g., ‘%,’ ‘$,’ etc.) or fake data) from the transcript 314 and transmits the redacted transcript as the de-identified transcript 337 outside the security boundary 130 for further processing (e.g., to the cloud computing device(s) 140).

As shown, the audio data de-Identification 328 also receives as input the transcript metadata 318 (and audio stream 232) and outputs the de-identified audio stream 338. Similar to the processing by the transcript de-identification 327, for each identified entity that includes PII or sensitive information, the audio data de-identification 328 can redact that corresponding portion of the audio stream 232 based on the timestamps for such identified entity (e.g., replacing that portion of the audio stream 232 with beep(s) or silence) indicated by the transcript metadata 318. As shown, the audio data de-Identification 328 transmits the de-identified audio stream 338 outside the security boundary 130 for further processing (e.g., to the cloud computing device(s) 140).

In some embodiments, the audio data de-identification 328 can perform de-identification of PII or sensitive information using cross-stream information. For example, the audio data de-identification 328 can use PII or sensitive information identified in a first audio stream 232 for redaction to identify the same PII or sensitive information in a second audio stream 232 and accordingly redact such PII or sensitive information from the second audio stream 232 or the associated transcript(s).

Also as shown, the identity-specific audio metadata extraction 330 receives as input the audio stream 232. More specifically, given the audio stream 232, the identity-specific audio metadata extraction 330 can generate voiceprints for specific persons (e.g., such as HCP(s) 204 and/or patient 203 and/or other personnel). Each such voiceprint is a representation of the unique vocal characteristics of a specific person. The identity-specific audio metadata extraction 330 can be implemented using AL/ML models (e.g., a deep neutral network (DNN)). The identity-specific audio feature extraction 310 can also generate affective voice features (also known as emotional prosody or sentiment) associated with these person-specific voiceprints. The identity-specific audio metadata extraction 330 can store these person-specific voiceprints (and, in some embodiments, associated affective voice features) as identity-specific audio metadata 331 locally in the data store 120, e.g., to be used to identify the same persons when their voices are captured in the future.

As shown, the identity-neutral audio metadata extraction 329 can also take as input the audio stream 232. Specifically, given the audio stream 232, the identity-neutral audio feature extraction 311 can generate non-person specific voice features, such as affective voice features (also known as emotional prosody or sentiment). Such an operation can be implemented based on AL/ML model(s) (e.g., a DNN). Such non-person specific voice features (shown as the identity-neutral audio features 333) (and the associated timestamp information), are transmitted as part of the identity-neutral audio metadata 339 outside the security boundary 130 for further processing (e.g., to the cloud computing device(s) 140).

The identity-neutral audio metadata extraction 329 can also take as input the transcript metadata 318. Given the transcript metadata 318, the identity-neutral audio metadata extraction 329 can generate metadata that represents various types of events. Such an operation can be implemented using AL/ML model(s) (e.g., large language model(s) (LLM) and/or event models). In a first example, the identity-neutral audio metadata extraction 329 can identify an HCP 204 (e.g., a surgeon) giving instructions. In a second example, the identity-neutral audio metadata extraction 329 can identify an HCP 204 (e.g., a nurse or a surgeon) asking a question. In a third example, the identity-neutral audio metadata extraction 329 can identify an HCP 204 (e.g., a nurse) having a conversation with another HCP (e.g., a nurse). It should be understood that the examples of events above are illustrative and that the audio events in the MFR 102 are innumerable. As shown, identity-neutral audio metadata extraction 329 transmits such events (and the associated timestamp information) as part of the identity-neutral audio metadata 339 outside the security boundary 130 for further processing (e.g., to the cloud computing device(s) 140).

In some embodiments, the identity-neutral audio metadata extraction 329 can associate the extracted identity-neutral audio metadata 339 with one or more of the following information: data identifying the type of a given surgical procedure, data identifying the MFR 102, data identifying the medical facility A.

As shown, the ECD 120 can receive the other data streams 234 (described above). In some cases, the other data streams 234 do not include PII information or sensitive information. As such, the ECD 120 transmits the other data streams 234 outside the security boundary 130 without any local processing at the ECD 120 (e.g., to the cloud computing device(s) 140). In other cases, the other data streams 234 may include PII information or sensitive information. In those cases, the PII or sensitive information can be redacted using one or more suitable methods.

In some embodiments, all or a portion of the functionality implemented at the CCD 140 that performs further processing based on the de-identified data, as described below in FIG. 4, can also be implemented at the ECD 120 in various embodiments. In some embodiments, the computations performed at the ECD 120 or CCD 140 can be performed by certain sensors (e.g., visual sensors 220, sound sensors 222, and other sensors 224) in the MFR 102 that are equipped for performing such computations.

### Cloud Computing Device

FIG. 4 is a more detailed illustration of the CCD 140 of FIG. 1, according to various embodiments. As shown, the CCD 140 includes various software applications (or any other forms of computer-executable instructions) (e.g., stored in a system memory (not shown)). Such software applications can include a ML models refiner 422, a machine learning inference engine 420, and an analytics generator 424. In operation, such software applications above can execute on one or more processors (not shown) (e.g., CPU(s) or GPU(s)) to perform one or more operations.

As shown, the ML models refiner 422 can receive, as input, the de-identified video stream 332, identity-neutral video metadata 334, de-identified transcript 337, de-identified audio stream 338, identity-neutral audio metadata 339, and other data stream 234 (which are collectively herein referred to as edge computing device (ECD) output) from the ECD 120 at medical facility A. It should be understood that the CCD(s) 140 can receive the ECD output from one or more medical facilities that are illustrated as medical facilities A-N.

Given the ECD output, the ML models refiner 422 can perform various operations to improve the ML models that were used to generate the ECD output. For example, the de-identified video stream 332 can be combined with its associated detection metadata 330, as a combined video stream, and presented to a user (also referred to as an annotator or labeler). As discussed above, the detection metadata 330 describes the detected objects and persons in the video stream 230, which are predictions of the ML model(s) used by object detection 302 (also referred to as object detection 302 ML model(s)). The combined video stream can present indications of the detected objects and persons visually to an annotator to allow for verifying whether the detection of an object or a person in the unredacted portion(s) of a given video stream was performed correctly. For example, if the detection was incorrect, the annotator can label the detection as incorrect. If the detection was correct, the annotator can label the detection as correct. In such a manner, such a data labeling process generates labels for the original predictions of the object detection 302 ML model(s). Such a data labeling process can be implemented using ML training platforms, such as the Google Cloud ML training platform. The ML models refiner 422 can further train and improve the object detection 302 ML model(s) based on the generated labels.

The ML models refiner 422 can further train and improve the ML model(s) used to detect the event(s) that are part of the identity-neutral video metadata 334. More specifically, as discussed above, event(s) are detected from a video frame or a series of video frames in the video stream 230 using ML model(s) (also referred to as event detection ML model(s)). Such detected event(s) can be combined with the de-identified video stream 332 and presented to an annotator for data labeling. Based on the generated labels, the ML models refiner 422 can further train and improve such event detection ML model(s).

Further, the ML models refiner 422 can train and improve the ML model(s) used to generate the de-identified transcript 337. More specifically, the de-identified audio stream 338 can be played back to an annotator while the de-identified transcript 337 is presented in synchronized manner to the annotator (e.g., synchronized based on timestamp information in both 337 and 338). In such a manner, the annotator can label whether the unredacted portion(s) of the de-identified transcript 337 was correctly generated. Based on the generated labels, the ML models refiner 422 can further train and improve such ML model(s).

Further, the ML models refiner 422 can train and improve the ML model(s) used to detect events that are part of the identity-neutral audio metadata 339. More specifically, the de-identified audio stream 338 can be played back to an annotator while the detected event(s) are presented in synchronized manner to the annotator (e.g., synchronized based on timestamp information). In such a manner, the annotator can label whether the detected event(s) were correctly generated. Based on the generated labels, the ML models refiner 422 can further train and improve such ML model(s).

The ML models refiner 422 can further train and improve the ML model(s) used to detect the non-person specific voice features in the identity-neutral audio metadata 339. More specifically, as discussed above, non-person specific voice features are detected from the audio stream 232. Such detected voice features can be presented synchronously with the playback of the de-identified audio stream 338 to an annotator for data labeling. Based on the generated labels, the ML models refiner 422 can further train and improve such ML model(s).

As shown, the ML models refiner 422 can transmit one or more of the above improved ML models, as updated ML model(s) 124, back to the ECD 120 in medical facility A (and, in some embodiments, other ECDs 120 in other medical facilities). In such a manner, given the updated ML mode(s) 124, the ECD 120 can improve the accuracy of various operations performed by one or more of the object detection 320, identity-neutral video metadata extraction 324, speech recognition 308, transcript processor 316 (which can include named entity recognition (NER) 316), identity-neutral audio metadata extraction 329. The process for refining and deploying refined ML models as described herein can be implemented using MLOps service(s), such as Google Cloud Platform services.

It should be understood that the above-described re-training operations of the ML models refiner 422 are illustrative only, and the ML models refiner 422 (and, in some embodiments, in conjunction with the ECD 120) can implement such operations using any suitable methods, such as federated training, active training, centralized training, de-centralized training, and the like.

As shown, the machine learning inference engine 420 can also receive, as input, the ECD output. Given the ECD output, the machine learning inference engine 420 can perform various operations to generate further inference(s). Because the ECP output is generated from or originates from various types of sensor data streams at a stream level (e.g., visual data stream, audio data stream, other sensor data streams), further inferences can be derived based on correlation(s) (or lack of correlation(s)) between these data streams. For instance, detected event(s) in the identity-neutral video metadata 334 can be correlated with the de-identified transcript 337 based the timestamp information. In a first example, based on instructions being detected in the de-identified transcript 337 during a detected visual event of an HCP 204 preparing a patient's skin for a surgical procedure, the machine learning inference engine 420 can infer that the instructions are related to the detected visual event. In a second example, based on a question being detected in the de-identified transcript 337 during a detected visual event of a conversation between two HCPs during a stage of a surgical procedure, the machine learning inference engine 420 can infer that the question is related to that stage of the surgical procedure. In a third example, no mention of a missing head cover is detected in the de-identified transcript 337 while a visual event of an HCP 204 missing a head cover is detected in all the frames of the video stream 230. In such a case, the machine learning inference engine 420 can infer from the lack of correlation to indicate no one noticed the missing head cover and/or no one addressed the event. It should be understood that the further inferences above are illustrative only and such further inferences are innumerable. In some embodiments, to further safeguard the ECD output, the machine learning inference engine 420 can delete the timestamp information, e.g., after a period of time or once the process of deriving the above further inferences completes.

As shown, the machine learning inference engine 420 can send its output to the analytics generator 424 for further processing. For example, the above-mentioned further inferences from various medical facilities A-N can be aggregated to generate analytics related to operational practices at all these medical facilities and/or a subset of these medical facilities (e.g., based on geographical area boundaries).

Similarly, as shown, the analytics generator 424 can also receive the ECD output and further process the output. For example, detected event(s) in the identity-neutral video metadata 334 from various medical facilities A-N can be aggregated to generate analytics related to operational practices at all these medical facilities and/or a subset of these medical facilities (e.g., based on geographical area boundaries). Given the generated analytics, the CCD(s) 140 can store the analytics (e.g., to be access by end users). For example, as shown, the user device 142 can communicate with the CCD(s) 140 (e.g., via a user interface, such as a web interface) to access the analytics.

FIG. 5 is a flow diagram of method steps for the edge computing device (ECD) 120 in FIGS. 1-3, according to various embodiments. Although the method steps are described in conjunction with FIGS. 1-3, persons skilled in the art will understand that any system configured to perform the method steps in any order falls within the scope of the present disclosure.

As shown, a method 500 begins at step 502, where the ECD 120 receives a set of streams of sensor data captured in a set of medical facility rooms in a medical facility. In some embodiments, each stream of the set of streams of sensor data can be captured using one of the following sensors in the monitoring system 219, as described above in conjunction with FIG. 2. The sensors can include a visual sensor, an audio sensor, a temperature sensor, a humidity sensor, a vibration sensor, and an ambient light sensor.

At step 504, for each stream of the set of streams of sensor data, the ECD performs steps 506 and 508. At step 506, the ECD 120 generates a de-identified data stream at least based on redacting from the stream information that identifies one or more persons. In some embodiments, a stream of the set of streams of sensor data is a video stream captured by a visual sensor a medical facility room of the set of operation rooms. In such embodiments, generating a de-identified data stream can also be based on redacting from the stream sensitive information related to a human body, as described above in conjunction with FIG. 3. Further, in such embodiments, redacting from the stream information that identifies one or more persons can include redacting images of the persons, as described above in conjunction with FIG. 3. In some embodiments, a stream of the set of streams of sensor data is an audio stream captured by an audio sensor a medical facility room of the set of operation rooms. In such embodiments, redacting from the stream information that identifies one or more persons can include redacting personal identifying information (PII) information from identified text in the stream, as described above in conjunction with FIG. 3.

At step 508, the ECD 120 generates identity-neutral metadata associated with the de-identified data stream at least based on extracting from the stream information that describes the identified persons in a manner that does not reveal the identity of the identified persons. In some embodiments, a stream of the set of streams of sensor data is a video stream captured by a visual sensor in a medical facility room of the set of operation rooms. In such embodiments, extracting information that describes the identified persons in a manner that does not reveal the identity of the identified persons can include detecting events related to the identified persons from one or more video frames of the stream, as described above in conjunction with FIG. 3. Further, in such embodiments, extracting information that describes the identified persons in a manner that does not reveal the identity of the identified persons can include extracting a skeleton body structure for each of the identified persons in each video frame of the stream, as described above in conjunction with FIG. 3. In some embodiments, a stream of the set of streams of sensor data is an audio stream captured by an audio sensor in a medical facility room of the set of operation rooms. In such embodiments, extracting information that describes the identified persons in a manner that does not reveal the identity of the identified persons can include extracting from the stream non-person specific voiceprints, as described above in conjunction with FIG. 3.

At step 510, the ECD 120 transmits the de-identified data streams and the associated identity-neutral metadata for further processing.

In sum, techniques are disclosed for de-identifying sensor data captured in medical facility rooms while maintaining the key features of the data in an obfuscated manner. In the disclosed system, sensor data captured in a medical facility room is processed in two phases. In a first phase, data de-identification operations are performed on the sensor data at an edge computing device that is within the security boundaries (e.g., firewall) of the medical facility or other healthcare service provider affiliated with the medical facility room. In a second phase, the de-identified sensor data is transmitted to one or more cloud services for further processing, including machine learning operations and data analytics operations.

In the de-identification phase, the edge computing device receives a video data stream from a medical facility room and performs object detection operations on the video data steam to identify persons and objects in the video data stream. Portions of the video stream that include persons or any personal identifying information (PII) associated with those persons are redacted (e.g., the pixels are deleted). Once redacted, any identifiable characteristics of the person cannot be discerned from the video stream. Further, the edge computing device generates metadata describing any persons that were identified in the video stream in an identity-neutral way, such that the underlying identity of the person cannot be discerned from the identity-neutral metadata. Metadata may describe the event(s) that occurred between different identified persons and/or between identified person(s) and identified objects. The edge computing device transmits the de-identified video data and the identity-neutral video metadata to a cloud computing device for generating data analytics and training Al/ML models.

Further, in the de-identification phase, the edge computing device receives an audio data stream from the medical facility room and performs speech recognition operation(s) on the audio data stream to convert the audio data stream to text (e.g., a transcript). Portions of the transcript that includes PII information are redacted (e.g., replaced with special characters). The transcript may include timestamp information for its text such that the portion of the audio data stream that corresponds to a redacted portion of the transcript may be identified and redacted (e.g., replaced with beep(s) or silence). Furthermore, the edge computing device performs audio feature operation(s) on the audio data stream to generate metadata that describes any audio features present in the audio data stream in an identity-neutral way, such that the underlying identify of the speaker cannot be discerned from the identity-neutral metadata. Metadata may describe affective and/or prosodic features of a person's voice. The edge computing device transmits the de-identified transcript(s) and audio data, and the identity-neutral audio metadata to a cloud computing device for generating data analytics and training Al/ML models.

At least one technical advantage of the disclosed techniques relative to the prior art is that the disclosed techniques can be effectively utilized to safeguard the privacy of healthcare data without sacrificing the ability to generate rich data analytics and/or train robust predictive models based on that data or vice versa. In particular, given a data stream captured in a medical facility room, the disclosed techniques can redact confidential and sensitive information from the data stream while at the same time generating identity-neutral metadata associated with the data stream. Such identity-neutral metadata preserves important data features in the data stream without revealing the underlying confidential and sensitive information in the data stream. Such a technique enables further processing at downstream applications to train predictive models (e.g., Al/ML models) and extract rich analytics. These technical advantages represent one or more technological improvements over prior art approaches.

Any and all combinations of any of the claim elements recited in any of the claims and/or any elements described in this application, in any fashion, fall within the contemplated scope of the present invention and protection.

1. In various embodiments, a computer-implemented method for de-identifying data captured in medical facility rooms comprises receiving, at one or more edge devices operating within a security boundary of a medical facility, a set of streams of sensor data captured in a set of medical facility rooms in the medical facility, for each stream of the set of streams of sensor data, generating, at the one or more edge devices, a de-identified data stream at least based on redacting from the stream information that identifies one or more persons, and generating, at the one or more edge devices, identity-neutral metadata associated with the de-identified data stream at least based on extracting from the stream information that describes the identified persons in a manner that does not reveal the identity of the identified persons; and transmitting the de-identified data streams and the associated identity-neutral metadata for further processing outside of the security boundary of the medical facility.

2. The computer-implemented method of clause 1, wherein each stream of the set of streams of sensor data is captured using one of the following sensors: a visual sensor, an audio sensor, a temperature sensor, a humidity sensor, a vibration sensor, or an ambient light sensor.

3. The computer-implemented method of clause 1 or 2, wherein a stream of the set of streams of sensor data comprises a video stream captured by a visual sensor in a medical facility room of the set of operation rooms, and wherein generating a de-identified data stream is further based on redacting from the stream sensitive information related to a human body.

4. The computer-implemented method of any of clauses 1-3, wherein a stream of the set of streams of sensor data comprises a video stream captured by a visual sensor in a medical facility room of the set of operation rooms, and wherein redacting from the stream information that identifies one or more persons comprises redacting images of the identified persons.

5. The computer-implemented method of any of clauses 1-4, wherein a stream of the set of streams of sensor data comprises an audio stream captured by an audio sensor in a medical facility room of the set of operation rooms, and wherein redacting from the stream information that identifies one or more persons comprises redacting personal identifying information (PII) information from identified text in the stream.

6. The computer-implemented method of any of clauses 1-5, wherein a stream of the set of streams of sensor data comprises a video stream captured by a visual sensor in a medical facility room of the set of operation rooms, and wherein extracting from the stream information that describes the identified persons in a manner that does not reveal the identity of the identified persons comprises detecting events related to the identified persons in one or more video frames of the stream.

7. The computer-implemented method of any of clauses 1-6, wherein a stream of the set of streams of sensor data comprises a video stream captured by a visual sensor in a medical facility room of the set of operation rooms, and wherein extracting from the stream information that describes the identified persons in a manner that does not reveal the identity of the identified persons comprises extracting a skeleton body structure for each of the identified persons in each video frame of the stream.

8. The computer-implemented method of any of clauses 1-7, wherein a stream of the set of streams of sensor data comprises an audio stream captured by an audio sensor in a medical facility room of the set of operation rooms, and wherein extracting from the stream information that describes the identified persons in a manner that does not reveal the identity of the identified persons comprises extracting from the stream non-person specific voiceprints.

9. The computer-implemented method of any of clauses 1-8, the method further comprising, for each stream of the set of streams of sensor data, generating identity-specific metadata based on extracting from the stream information that identifies a specific person.

10. The computer-implemented method of any of clauses 1-9, wherein the stream comprises a video stream captured by a visual sensor in a medical facility room of the set of operation rooms, and wherein extracting from the stream information that identifies a specific person comprises generating a de-identified visual model of the person based on images of the person.

11. The computer-implemented method of any of clauses 1-10, wherein the stream comprises an audio stream captured by an audio sensor in a medical facility room of the set of operation rooms, and wherein extracting from the stream information that identifies a specific person comprises generating a person-specific voiceprint of the person.

12. In various embodiments, one or more non-transitory computer-readable storage media include instructions that, when executed by one or more processing units, cause the one or more processing units to perform steps for de-identifying data captured in medical facility rooms, the steps comprising: receiving, at one or more edge devices operating within a security boundary of a medical facility, a set of streams of sensor data captured in a set of medical facility rooms in the medical facility; for each stream of the set of streams of sensor data, generating, at the one or more edge devices, a de-identified data stream at least based on redacting from the stream information that identifies one or more persons, and generating, at the one or more edge devices, identity-neutral metadata associated with the de-identified data stream at least based on extracting from the stream information that describes the identified persons in a manner that does not reveal the identity of the identified persons; and transmitting the de-identified data streams and the associated identity-neutral metadata for further processing outside of the security boundary of the medical facility.

13. The one or more non-transitory computer readable media of clause 13, wherein a stream of the set of streams of sensor data comprises a video stream captured by a visual sensor in a medical facility room of the set of operation rooms, and wherein generating a de-identified data stream is further based on redacting from the stream sensitive information related to a human body.

14. The one or more non-transitory computer readable media of clause 12 or 13, wherein a stream of the set of streams of sensor data comprises a video stream captured by a visual sensor in a medical facility room of the set of operation rooms, and wherein redacting from the stream information that identifies one or more persons comprises redacting images of the identified persons.

15. The one or more non-transitory computer readable media of any of clauses 12-14, wherein a stream of the set of streams of sensor data comprises an audio stream captured by an audio sensor in a medical facility room of the set of operation rooms, and wherein redacting from the stream information that identifies one or more persons comprises redacting personal identifying information (PII) information from identified text in the stream.

16. The one or more non-transitory computer readable media of any of clauses 12-15, wherein a stream of the set of streams of sensor data comprises a video stream captured by a visual sensor in a medical facility room of the set of operation rooms, and wherein extracting from the stream information that describes the identified persons in a manner that does not reveal the identity of the identified persons comprises detecting events related to the identified persons in one or more video frames of the stream.

17. The one or more non-transitory computer readable media of any of clauses 12-16, wherein a stream of the set of streams of sensor data comprises a video stream captured by a visual sensor in a medical facility room of the set of operation rooms, and wherein extracting from the stream information that describes the identified persons in a manner that does not reveal the identity of the identified persons comprises extracting a skeleton body structure for each of the identified persons in each video frame of the stream.

18. The one or more non-transitory computer readable media of any of clauses 12-17, wherein a stream of the set of streams of sensor data comprises an audio stream captured by an audio sensor in a medical facility room of the set of operation rooms, and wherein extracting from the stream information that describes the identified persons in a manner that does not reveal the identity of the identified persons comprises extracting from the stream non-person specific voiceprints.

19. The one or more non-transitory computer readable media of any of clauses 12-18, wherein the steps further comprise for each stream of the set of streams of sensor data, generating identity-specific metadata based on extracting from the stream information that identifies a specific person.

20. In various embodiments, a system, comprises: a memory that stores instructions, and a processor that is coupled to the memory and, when executing the instructions, is configured to: receiving, at one or more edge devices operating within a security boundary of a medical facility, a set of streams of sensor data captured in a set of medical facility rooms in the medical facility; for each stream of the set of streams of sensor data, generating, at the one or more edge devices, a de-identified data stream at least based on redacting from the stream information that identifies one or more persons, and generating, at the one or more edge devices, identity-neutral metadata associated with the de-identified data stream at least based on extracting from the stream information that describes the identified persons in a manner that does not reveal the identity of the identified persons; and transmitting the de-identified data streams and the associated identity-neutral metadata for further processing outside of the security boundary of the medical facility.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

Aspects of the present embodiments may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “module,” a “system,” or a “computer.” In addition, any hardware and/or software technique, process, function, component, engine, module, or system described in the present disclosure may be implemented as a circuit or set of circuits. Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Aspects of the present disclosure are described above with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine. The instructions, when executed via the processor of the computer or other programmable data processing apparatus, enable the implementation of the functions/acts specified in the flowchart and/or block diagram block or blocks. Such processors may be, without limitation, general purpose processors, special-purpose processors, application-specific processors, or field-programmable gate arrays.

The flowchart and block diagrams in the figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

While the preceding is directed to embodiments of the present disclosure, other and further embodiments of the disclosure may be devised without departing from the basic scope thereof, and the scope thereof is determined by the claims that follow.

What is claimed is:

1. A computer-implemented method for de-identifying data captured in medical facility rooms, the method comprising:
receiving, at one or more edge devices operating within a security boundary of a medical facility, a set of streams of sensor data captured in a set of medical facility rooms in the medical facility;
for each stream of the set of streams of sensor data,
generating, at the one or more edge devices, a de-identified data stream at least based on redacting from the stream information that identifies one or more persons, and
generating, at the one or more edge devices, identity-neutral metadata associated with the de-identified data stream at least based on extracting from the stream information that describes the identified persons in a manner that does not reveal the identity of the identified persons; and
transmitting the de-identified data streams and the associated identity-neutral metadata for further processing outside of the security boundary of the medical facility.

2. The computer-implemented method of claim 1, wherein each stream of the set of streams of sensor data is captured using one of the following sensors: a visual sensor, an audio sensor, a temperature sensor, a humidity sensor, a vibration sensor, or an ambient light sensor.

3. The computer-implemented method of claim 1, wherein a stream of the set of streams of sensor data comprises a video stream captured by a visual sensor in a medical facility room of the set of operation rooms, and wherein generating a de-identified data stream is further based on redacting from the stream sensitive information related to a human body.

4. The computer-implemented method of claim 1, wherein a stream of the set of streams of sensor data comprises a video stream captured by a visual sensor in a medical facility room of the set of operation rooms, and wherein redacting from the stream information that identifies one or more persons comprises redacting images of the identified persons.

5. The computer-implemented method of claim 1, wherein a stream of the set of streams of sensor data comprises an audio stream captured by an audio sensor in a medical facility room of the set of operation rooms, and wherein redacting from the stream information that identifies one or more persons comprises redacting personal identifying information (PII) information from identified text in the stream.

6. The computer-implemented method of claim 1, wherein a stream of the set of streams of sensor data comprises a video stream captured by a visual sensor in a medical facility room of the set of operation rooms, and wherein extracting from the stream information that describes the identified persons in a manner that does not reveal the identity of the identified persons comprises detecting events related to the identified persons in one or more video frames of the stream.

7. The computer-implemented method of claim 1, wherein a stream of the set of streams of sensor data comprises a video stream captured by a visual sensor in a medical facility room of the set of operation rooms, and wherein extracting from the stream information that describes the identified persons in a manner that does not reveal the identity of the identified persons comprises extracting a skeleton body structure for each of the identified persons in each video frame of the stream.

8. The computer-implemented method of claim 1, wherein a stream of the set of streams of sensor data comprises an audio stream captured by an audio sensor in a medical facility room of the set of operation rooms, and wherein extracting from the stream information that describes the identified persons in a manner that does not reveal the identity of the identified persons comprises extracting from the stream non-person specific voiceprints.

9. The computer-implemented method of claim 1, the method further comprising: for each stream of the set of streams of sensor data, generating identity-specific metadata based on extracting from the stream information that identifies a specific person.

10. The computer-implemented method of claim 9, wherein the stream comprises a video stream captured by a visual sensor in a medical facility room of the set of operation rooms, and wherein extracting from the stream information that identifies a specific person comprises generating a de-identified visual model of the person based on images of the person.

11. The computer-implemented method of claim 9, wherein the stream comprises an audio stream captured by an audio sensor in a medical facility room of the set of operation rooms, and wherein extracting from the stream information that identifies a specific person comprises generating a person-specific voiceprint of the person.

12. One or more non-transitory computer-readable storage media including instructions that, when executed by one or more processing units, cause the one or more processing units to perform steps for de-identifying data captured in medical facility rooms, the steps comprising:
receiving, at one or more edge devices operating within a security boundary of a medical facility, a set of streams of sensor data captured in a set of medical facility rooms in the medical facility;
for each stream of the set of streams of sensor data,
generating, at the one or more edge devices, a de-identified data stream at least based on redacting from the stream information that identifies one or more persons, and
generating, at the one or more edge devices, identity-neutral metadata associated with the de-identified data stream at least based on extracting from the stream information that describes the identified persons in a manner that does not reveal the identity of the identified persons; and
transmitting the de-identified data streams and the associated identity-neutral metadata for further processing outside of the security boundary of the medical facility.

13. The one or more non-transitory computer readable media of claim 12, wherein a stream of the set of streams of sensor data comprises a video stream captured by a visual sensor in a medical facility room of the set of operation rooms, and wherein generating a de-identified data stream is further based on redacting from the stream sensitive information related to a human body.

14. The one or more non-transitory computer readable media of claim 12, wherein a stream of the set of streams of sensor data comprises a video stream captured by a visual sensor in a medical facility room of the set of operation rooms, and wherein redacting from the stream information that identifies one or more persons comprises redacting images of the identified persons.

15. The one or more non-transitory computer readable media of claim 12, wherein a stream of the set of streams of sensor data comprises an audio stream captured by an audio sensor in a medical facility room of the set of operation rooms, and wherein redacting from the stream information that identifies one or more persons comprises redacting personal identifying information (PII) information from identified text in the stream.

16. The one or more non-transitory computer readable media of claim 12, wherein a stream of the set of streams of sensor data comprises a video stream captured by a visual sensor in a medical facility room of the set of operation rooms, and wherein extracting from the stream information that describes the identified persons in a manner that does not reveal the identity of the identified persons comprises detecting events related to the identified persons in one or more video frames of the stream.

17. The one or more non-transitory computer readable media of claim 12, wherein a stream of the set of streams of sensor data comprises a video stream captured by a visual sensor in a medical facility room of the set of operation rooms, and wherein extracting from the stream information that describes the identified persons in a manner that does not reveal the identity of the identified persons comprises extracting a skeleton body structure for each of the identified persons in each video frame of the stream.

18. The one or more non-transitory computer readable media of claim 12, wherein a stream of the set of streams of sensor data comprises an audio stream captured by an audio sensor in a medical facility room of the set of operation rooms, and wherein extracting from the stream information that describes the identified persons in a manner that does not reveal the identity of the identified persons comprises extracting from the stream non-person specific voiceprints.

19. The one or more non-transitory computer readable media of claim 12, wherein the steps further comprise for each stream of the set of streams of sensor data, generating identity-specific metadata based on extracting from the stream information that identifies a specific person.

20. A system, comprising:
a memory that stores instructions, and
a processor that is coupled to the memory and, when executing the instructions, is configured to:
receiving, at one or more edge devices operating within a security boundary of a medical facility, a set of streams of sensor data captured in a set of medical facility rooms in the medical facility;
for each stream of the set of streams of sensor data,
generating, at the one or more edge devices, a de-identified data stream at least based on redacting from the stream information that identifies one or more persons, and
generating, at the one or more edge devices, identity-neutral metadata associated with the de-identified data stream at least based on extracting from the stream information that describes the identified persons in a manner that does not reveal the identity of the identified persons; and
transmitting the de-identified data streams and the associated identity-neutral metadata for further processing outside of the security boundary of the medical facility.
